(19) United States
(12) Patent Application Publication
(10) Pub. No.: US 2025/0235257 A1
(43) Pub. Date: Jul. 24, 2025

(54) HIGH TEMPERATURE SPACER FOR JAW MEMBER AND METHOD FOR MANUFACTURING SAME

(71) Applicant: Covidien LP, Mansfield, MA (US)

(72) Inventors: James D. ALLEN, IV, Boulder, CO (US); Kenneth E. NETZEL, Boulder, CO (US)

(73) Assignee: Covidien LP, Mansfield, MA (US)

(21) Appl. No.: 18/852,716

(22) PCT Filed: Mar. 31, 2023
(86) PCT No.: PCT/IB2023/053242

§ 371 Date: Sep. 30, 2024

Related U.S. Application Data
(60) Provisional application No. 63/325,812, filed on Mar. 31, 2022.

(51) Int. Cl. A61B 18/14; A61B 17/00; A61B 18/00

(52) U.S. Cl. CPC A61B 18/1445; A61B 2017/00526; A61B 2018/00077; A61B 2018/00101; A61B 2018/00601; A61B 2018/0063; A61B 2018/146

(57) ABSTRACT

A jaw member for a surgical instrument includes an insulative spacer configured to retain a thermal cutter assembly therein. A jaw support is configured to encapsulate the insulative spacer and securely engage the same. An overmold is configured to encapsulate and secure the insulative spacer and jaw support. The insulative spacer is made from a material having a high temperature resistance and low thermal conductivity to reduce heat transfer to the overmold during activation of the thermal cutter assembly.

### FIELD

The present disclosure relates to surgical instruments and, more particularly, to electrosurgical instruments for sealing and cutting tissue, and methods of manufacturing same.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a jaw member for a surgical instrument which includes an insulative spacer configured to retain a thermal cutter assembly therein. A jaw support is configured to partially or fully encapsulate the insulative spacer and securely engage the insulative spacer therein. An overmold is configured to encapsulate and secure the insulative spacer and the jaw support, wherein the insulative spacer is made from a material having a high temperature resistance and low thermal conductivity to reduce heat transfer to the overmold during activation of the thermal cutter assembly.

In aspects in accordance with the present disclosure, the insulative spacer is made from a machinable ceramic material.

In aspects in accordance with the present disclosure, the insulative spacer is made from polybenzimidazole (PBI).

In aspects in accordance with the present disclosure, the thermal cutter assembly is retained within the insulative spacer by a high temperature adhesive.

In aspects in accordance with the present disclosure, the jaw members includes a second overmold disposed between the jaw support and the insulative spacer.

In aspects in accordance with the present disclosure, the insulative spacer includes a plurality of spacer divots configured to enhance operative engagement with the overmold.

Provided in accordance with aspects of the present disclosure is a method of assembling a jaw member which includes securing a thermal cutter assembly within a high temperature insulative spacer having a low conductivity; encapsulating the insulative spacer with a jaw support, the jaw support including one or more proximal flanges for operably engaging an opposing jaw member; and overmolding the jaw support and insulative spacer to encapsulate and secure both the jaw support and the insulative spacer.

In aspects in accordance with the present disclosure, the insulative spacer includes a plurality of spacer divots configured to enhance operative engagement with the overmold.

In aspects in accordance with the present disclosure, prior to encapsulating the insulative spacer with the jaw support, the method includes overmolding the insulative spacer to encapsulate and secure a portion of the insulative spacer.

In aspects in accordance with the present disclosure, prior to encapsulating the insulative spacer with the jaw support the method includes engaging a wire to the insulative spacer.

In aspects in accordance with the present disclosure, engaging the wire to the insulative spacer includes a wire clip.

In aspects in accordance with the present disclosure, the method further includes depositing a conductive material atop a tissue engaging surface of the insulative spacer. In other aspects in accordance with the present disclosure, the method further includes depositing a conductive material atop a tissue engaging surface of the insulative spacer and the wire clip to form an electrical connection between the conductive material and the wire.

In aspects in accordance with the present disclosure, prior to encapsulating the insulative spacer with the jaw support, the method includes overmolding the insulative spacer to encapsulate and secure a portion of the insulative spacer and engaging a wire to the insulative spacer. In other aspects in accordance with the present disclosure, engaging the wire to the insulative spacer includes a wire clip. In yet other aspects in accordance with the present disclosure, the method further includes depositing a conductive material atop a tissue engaging surface of the insulative spacer.

In aspects in accordance with the present disclosure, the method further includes depositing a conductive material atop a tissue engaging surface of the insulative spacer and the wire clip to form an electrical connection between the conductive material and the wire. In other aspects in accordance with the present disclosure, the conductive material is selected from the group consisting of aluminum, copper, chromium, titanium, stainless steel, nickel, chrome, tin, platinum, zinc, palladium, gold, nichrome, and ferritic iron-chromium-aluminum alloys.

In aspects in accordance with the present disclosure, the insulative spacer is made from a machinable ceramic material.

In aspects in accordance with the present disclosure, the insulative spacer is made from polybenzimidazole (PBI).

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of a distal end portion of the forceps of FIG. 1, wherein first and second jaw members of an end effector assembly of the forceps are disposed in a spaced-apart position exposing a thermal cutter assembly;

FIG. 5 is a perspective view of a distal end portion of the forceps of FIG. 1, wherein first and second jaw members of the end effector assembly of the forceps are disposed in a spaced-apart position and the thermal cutter assembly is separated therefrom exposing a slot defined in one of the jaw members;

FIG. 6A is a schematic view of the thermal cutter assembly in accordance with the present disclosure;

FIG. 6B is a schematic side view of the thermal cutter assembly in accordance with the present disclosure;

FIGS. 7A-7C are perspective views of one embodiment of a jaw member with a high temperature insulative spacer;

FIGS. 8A-8C are perspective views of another embodiment of a jaw member with a high temperature insulative spacer; and

FIGS. 9A and 9B are schematic views of various embodiments of a slot of a jaw member having a series of standoffs disposed therein configured to control the heat dissipation to surrounding jaw member structures.

### DETAILED DESCRIPTION

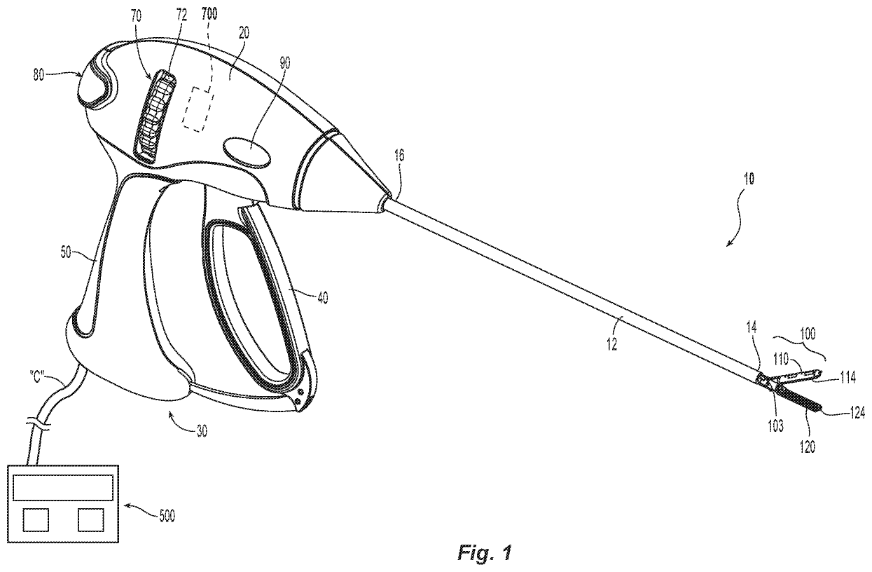

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

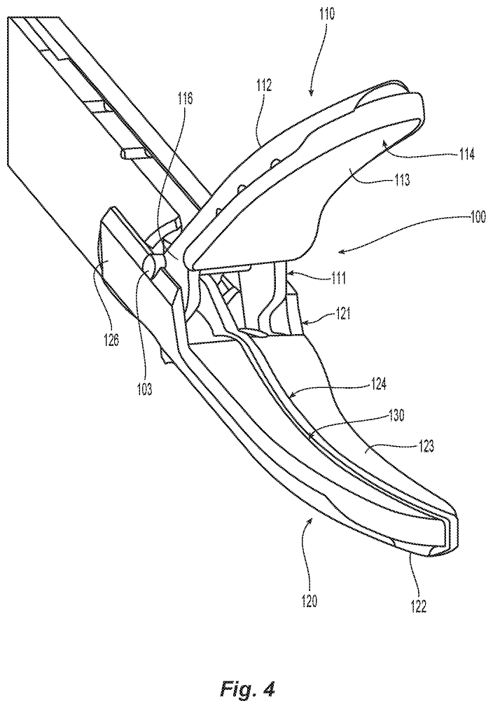

Forceps 10 includes a housing 20, a handle assembly 30, a rotating assembly 70, a first activation switch 80, a second activation switch 90, and an end effector assembly 100. As shown, end effector assembly 100 includes jaw members 110 and 120 configured for unilateral movement relative to one another. Bilateral movement of the jaw members 110, 120 is also envisioned. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue-treating surfaces 114, 124 of jaw members 110, 120, respectively, of end effector assembly 100 (see FIG. 4) to provide energy thereto.

First activation switch 80 is coupled to tissue-treating surfaces 114, 124 (FIG. 4) and the electrosurgical generator “G” for enabling the selective activation of the supply of energy to jaw members 110, 120 for treating, e.g., cauterizing, coagulating/desiccating, and/or sealing, tissue. Second activation switch (e.g., thumb switch 90) is coupled to thermal cutter assembly 130 of jaw member 120 (FIG. 4) and the electrosurgical generator “G” for enabling the selective activation of the supply of energy to thermal cutter assembly 130 for thermally cutting tissue. Second activation switch 90 may be actuated via any finger, in-line with handle, footswitch, etc.

Alternatively, a single activation switch may be utilized wherein the generator “G” sequentially seals and then cuts with a single actuation of the switch, e.g., switch 80. A “seal” may be indicated by an audible tone from the generator “G” and after a short or programmable delay the forceps 10 (or the generator algorithm) transitions into a cut cycle or cut “mode”. Again a “cut” may be represented by a different tone from the generator “G” or from the forceps 10.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue-treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

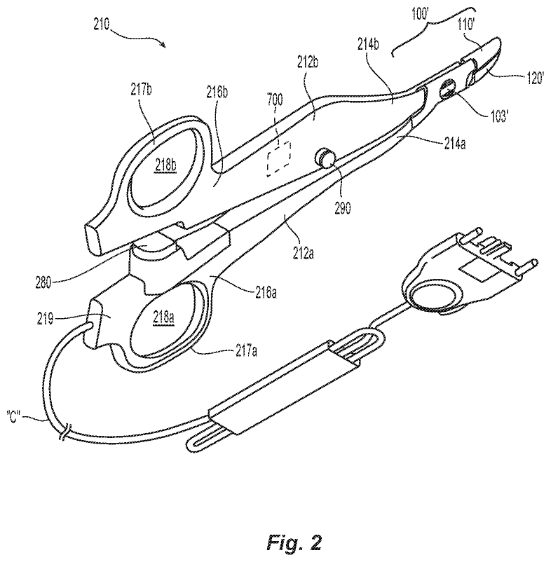

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 (similar to activation switch 80 discussed above) is provided for supplying energy to jaw members 110′, 120′ to treat tissue upon sufficient approximation of shaft members 212a, 212b, e.g., upon activation of first activation switch 280 via shaft member 212a. A second activation switch 290 (similar to second activation switch 90 discussed above) disposed on either or both of shaft members 212a, 212b is coupled to the thermal cutter element (not shown, similar to thermal cutter assembly 130 of jaw member 120 (FIG. 4)) of one of the jaw members 110′, 120′ of end effector assembly 100′ and to the electrosurgical generator “G” for enabling the selective activation of the supply of energy to the thermal cutter assembly 130 for thermally cutting tissue.

Alternatively, a single activation switch may be utilized wherein the generator “G” sequentially seals and then cuts with a single actuation of the switch, e.g., switch 280. A “seal” may be indicated by an audible tone from the generator “G” and after a short or programmable delay the forceps 210 (or the generator algorithm) transitions into a cut cycle or cut “mode”. Again a “cut” may be represented by a different tone from the generator “G” or from the forceps 210.

Jaw members 110′, 120′ define a curved configuration wherein each jaw member is similarly curved laterally relative to a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

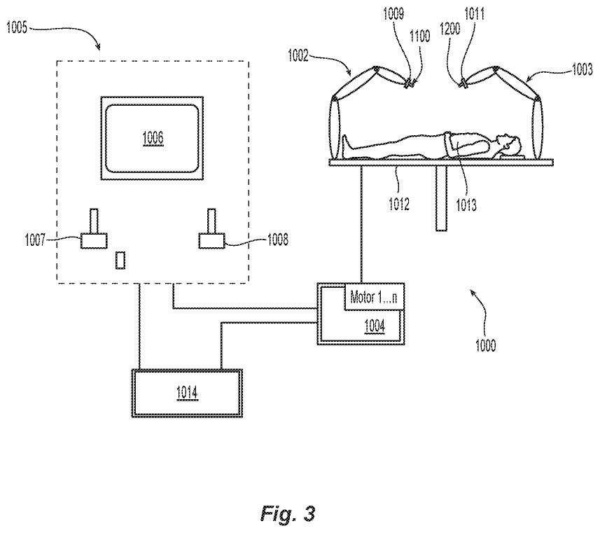

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assembly 100 (FIG. 4), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

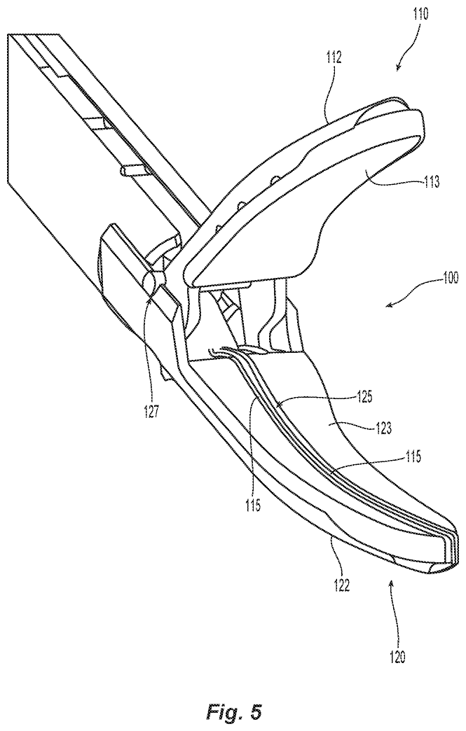

Turning to FIGS. 4-5, end effector assembly 100, as noted above, includes first and second jaw members 110, 120. Each jaw member 110, 120 may include a structural frame 111, 121, a jaw housing 112, 122, and a tissue-treating plate 113, 123 defining the respective tissue-treating surface 114, 124 thereof. Alternatively, only one of the jaw members, e.g., jaw member 120, may include structural frame 121, jaw housing 122, and tissue-treating plate 123 defining the tissue-treating surface 124. In such embodiments, the other jaw member, e.g., jaw member 110, may be formed as a single unitary body, e.g., a piece of conductive material acting as the structural frame 111 and jaw housing 112 and defining the tissue-treating surface 114.

An outer surface of the jaw housing 112, in such embodiments, may be at least partially coated with an electrically insulative material or may remain exposed. In embodiments, tissue-treating plates 113, 123 may be deposited onto jaw housings 112, 122 or jaw inserts (not shown) disposed within jaw housings 112, 122, e.g., via sputtering. Alternatively, tissue-treating plates 113, 123 may be pre-formed and engaged with jaw housings 112, 122 and/or jaw inserts (not shown) disposed within jaw housings 112, 122 via, for example, overmolding, adhesion, mechanical engagement, etc. Other methods of depositing the tissue-treating plates 113, 123 onto the jaw inserts are described in detail below.

Referring in particular to FIGS. 4 and 5, jaw member 110, as noted above, may be configured similarly as jaw member 120, may be formed as a single unitary body, or may be formed in any other suitable manner so as to define a structural frame 111 and a tissue-treating surface 114 opposing tissue-treating surface 124 of jaw member 120. Structural frame 111 includes a proximal flange portion 116 about which jaw member 110 is pivotably coupled to jaw member 120. In shaft-based or robotic embodiments, proximal flange portion 116 receives pivot 103 and which mounts atop flange 126 of jaw member 120 (FIG. 4) such that actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a screw-drive mechanism, etc.

For the purposes of further describing one or both of the jaw members 110, 120 (and 210, 220), each jaw member 110, 120 may include a longitudinally-extending insulative member 115 defined within a slot 125 extending along at least a portion of the length of tissue-treating surfaces 114, 124 (FIG. 5). Insulative member 115 may be transversely centered on either or both tissue-treating surfaces 114, 124 or may be offset relative thereto. As explained in more detail below with respect to jaw member 120, insulative member 115 may house and electrically and/or thermally isolate the thermal cutter assembly 130 separately activatable to cut tissue upon activation thereof. Further, insulative member 115 may be disposed, e.g., deposited, coated, etc., on tissue-treating surface 114, 124, may be positioned within the channel or recess defined within tissue-treating surface 114, 124, or may define any other suitable configuration.

Additionally, insulative member 115 may be substantially (within manufacturing, material, and/or use tolerances) coplanar with each respective tissue-treating surface 114, 124 may protrude from each respective tissue-treating surface 114, 124, may be recessed relative to each respective tissue-treating surface 114, 124 or may include different portions that are coplanar, protruding, and/or recessed relative to tissue-treating surfaces 114, 124. Moreover, insulative member 115 and thermal cutter assembly 130 may be curvilinear to follow the configuration of the jaw members 110, 120. Insulative member 115 may be formed from, for example, ceramic, parylene, glass, nylon, PTFE, or other suitable material(s) (including combinations of insulative and non-insulative materials).

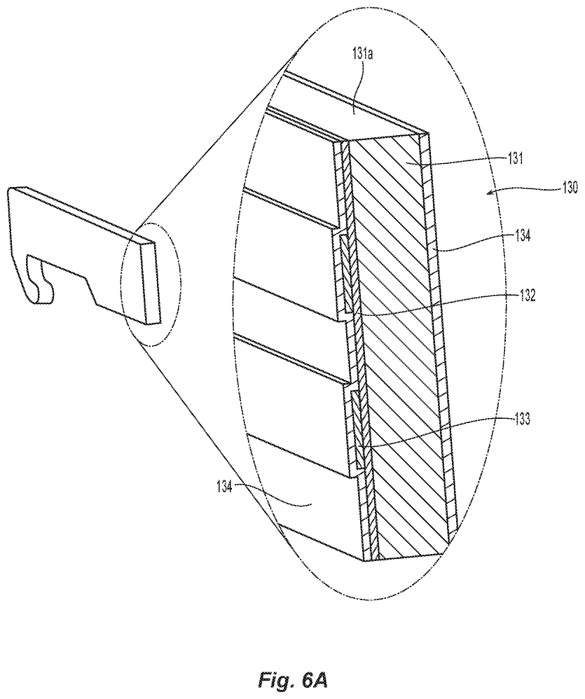

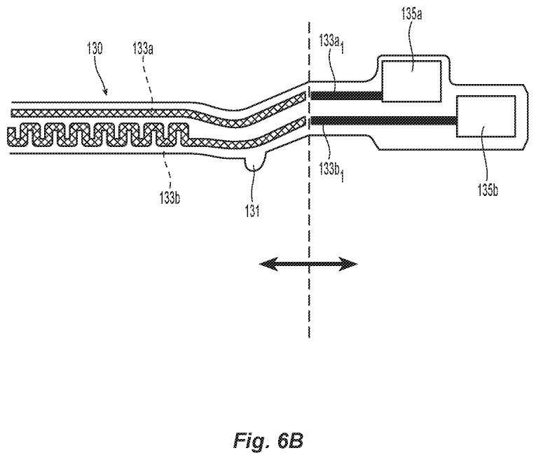

With reference to FIGS. 4 and 5, as noted above, jaw member 120 includes a structural frame 121, a jaw housing 122, and a tissue-treating plate 123 defining the tissue-treating surface 124 thereof. With reference also to FIG. 6A, details relating to the thermal cutter assembly 130 are generally defined to include the following elements (described internally to externally): substrate 131 or other internalized bendable metal structure that is both thermally and electrically conductive, e.g., stainless steel, aluminum, etc.; insulator 132 having generally electrically insulative properties and at least partially thermally conductive, e.g., sintered glass, alumina, plasma electrolytic oxidation (PEO anodize), Silica, etc.; resistive element 133 or any metal that is resistive but certain metals may have better thermal coefficients than others; and encapsulant 134 or an electrically insulative materials that is at least partially thermally conductive (may be the same or similar to the insulator). As explained below, the resistive element 133 may be deposited atop insulator 132 via sputtering or the like.

FIG. 6B shows a side view of thermal cutter assembly 130 and the electrical connections associated therewith. Generally, electrically conductive pads 135a, 135b connect to opposite ends 133a, 133b of resistive element 133 via traces 133a1, 133b1 which are electrically conductive traces (low resistance/low heat). As explained in detail below, resistive element 133 is configured to rapidly generate heat due to high resistive properties when electrical current is passed therethrough.

Structural frame 121 defines a proximal flange portion 126 and a distal body portion (not shown) extending distally from proximal flange portion 126. Proximal flange portion 126 is bifurcated to define a pair of spaced-apart proximal flange portion segments that receive proximal flange 111 of jaw member 110 therebetween and define aligned apertures 127 configured for receipt of pivot 103 therethrough/thereon to pivotably couple jaw members 110, 120 with one another (FIG. 5).

Jaw housing 122 of jaw member 120 is disposed about the distal body portion of structural frame 121, e.g., via overmolding, adhesion, mechanical engagement, etc., and supports tissue-treating plate 123 thereon, e.g., via overmolding, adhesion, mechanical engagement, depositing (such as, for example, via sputtering or thermal spraying), etc. Tissue-treating plate 123, as noted above, defines tissue-treating surface 124. Longitudinally-extending slot or channel 125 is defined through tissue-treating plate 123 and is positioned relative to jaw member 110 or an insulative member 115 disposed in vertical registration therewith when the jaw members 110 and 120 are in the approximated position (FIG. 5). The slot or channel 125 may be defined within an integrally-formed tissue-treating plate 123 or may be defined between two tissue-treating plates that, together, operate as a single treatment surface (not shown). Slot 125 may extend through at least a portion of jaw housing 122, a jaw insert (if so provided), and/or other components of jaw member 120 to enable receipt of thermal cutter assembly 130 at least partially within slot 125.

Thermal cutter assembly 130, more specifically, is disposed within longitudinally-extending slot 125 such that thermal cutter assembly 130 opposes jaw member 110 in the approximated position. Thermal cutter assembly 130 may be configured to contact jaw member 110 (or another insulative member 115 as mentioned above and as shown in FIG. 4) in the approximated position to regulate or contribute to regulation of a gap distance between tissue-treating surfaces 114, 124 in the approximated position. Alternatively or additionally, one or more stop members (not shown) associated with jaw member 110 and/or jaw member 120 may be provided to regulate the gap distance between tissue-treating surfaces 114, 124 in the approximated position.

Thermal cutter assembly 130 may be surrounded by the insulative member 115 disposed within slot 125 to electrically and/or thermally isolate thermal cutter assembly 130 from tissue-treating plate 123 (See FIG. 4 versus FIG. 5). As mentioned above, thermal cutter assembly 130 includes an encapsulant 134 that may act in conjunction with or in lieu of insulative member 115. Encapsulant 134 (and insulator 132 as shown in FIG. 6A) is configured cover the sides of the substrate 131 leaving the tissue facing edge 131a of the substrate 131 exposed. Thermal cutter assembly 130 and insulative member 115 may similarly or differently be substantially (within manufacturing, material, and/or use tolerances) coplanar with tissue-treating surface 124, may protrude from tissue-treating surface 124, may be recessed relative to tissue-treating surface 124, or may include different portions that are coplanar, protruding, and/or recessed relative to tissue-treating surface 124.

Turning back to the thermal cutter assembly 130 and the various methods of manufacturing the same, it is contemplated that the resistive element 133 of the thermal cutter assembly 130 may be manufactured in thin layers that are deposited atop (or otherwise) insulator 132 which is disposed atop substrate 131. For the purposes herein, the resistive element 133 will be described as being deposited onto insulator 132, knowing that insulator, in turn, may be disposed on one or both sides of substrate 131. For example, it is contemplated that resistive element 133 may be deposited onto the insulator 132 via one or more of the following manufacturing techniques: sputtering, thermal evaporation, thermal spraying, cathodic arcing, pulsed laser deposition, electron beam deposition. Other techniques may include: electroless strike or plating and electro-plating, shadow masking.

Utilizing one or more of these techniques provides a thin layer of thermally conductive resistive material which has the benefit of dissipating heat quickly compared to a traditional thermal cutter assembly 130. Other advantages of thin-layered, thermally conductive resistive elements 133 on the thermal cutter assembly 130 include: the ability to heat up quickly, the ability to require less energy to heat up and maintain heat during the cutting process, and the ability to cut tissue in a reduced timeframe compared to traditional electrical cutters.

Any one of the following materials (or combinations thereof) may be utilized as the resistive element 133: aluminum, copper, chromium, titanium, stainless steel, nickel, chrome, tin, platinum, palladium, gold, nichrome, and Kanthal®. It is contemplated that during manufacturing, combinations of materials may be utilized for a particular purpose or to achieve a particular result. For example, one material may be utilized as a base conductor with a second material used as an outer or inner conductor to act as the heating element. Additional techniques or materials may be added to act as thermal cutter assemblies 130 or resistive elements 133 such as those described with reference to U.S. patent application Ser. No. 16/785,347 filed Feb. 7, 2020, U.S. Provisional Patent Application Ser. No. 62/952,232 filed Dec. 21, 2019, U.S. patent application Ser. No. 16/838,551 filed Apr. 2, 2020, and U.S. patent application Ser. No. 16/518,016 filed Jul. 22, 2019, the entire contents of each of which being incorporated by reference herein.

In other embodiments, materials may be mixed during the application process. In some embodiments, the material used (e.g., Aluminum, copper etc.) may be thin and still promote a good cutting effect while other materials may have to be thicker to produce the same or similar cutting effect due to the particular material's level of electrical resistance. In this latter instance, a highly conductive base material may be utilized with the thinner, less conductive material more resistive material to produce a desired effect.

In embodiments, a biocompatible material (not shown) may be utilized to cover a non-biocompatible material. In other embodiments, the materials may be deposited (or otherwise disposed on insulator 132 in non-uniform layers while still allowing for transitions, e.g., side-to-side transitions. The materials could be deposited (or otherwise disposed on insulator 132) in an alternating fashion and more than one electrical circuit may be employed.

Examples of resistive elements 133 that may be used for thermal cutter assemblies 130 may include single layer resistive elements 133 in the range of about 0.1 micron to about 500 microns. A so-called “thick” film resistive element 133 would be about 30 microns and a “thin” film resistive element 133 would be about 1 micron. Non-conductive, electrically transparent, thermally transparent, or electrically and/or thermally porous materials may also be layered in a similar fashion atop, below or between the resistive elements 133. One or more of these materials may be layered atop the resistive elements 133 to complete the thermal cutter assembly 130 as mentioned above within a specified range.

Generally, tissue-treating plates 113, 123 are formed from an electrically conductive material, e.g., for conducting electrical energy therebetween for treating tissue, although tissue-treating plates 113, 123 may alternatively be configured to conduct any suitable energy, e.g., thermal, microwave, light, ultrasonic, etc., through tissue grasped therebetween for energy-based tissue treatment. As mentioned above, tissue-treating plates 113, 123 are coupled to activation switch 80 and electrosurgical generator “G” (FIG. 1) such that energy may be selectively supplied to tissue-treating plates 113, 123 and conducted therebetween and through tissue disposed between jaw members 110, 120 to treat tissue, e.g., seal tissue on either side and extending across thermal cutter assembly 130.

Thermal cutter assembly 130, on the other hand, is configured to connect to electrosurgical generator “G” (FIG. 1) and second activation switch 90 to enable selective activation of the supply of energy to thermal cutter assembly 130 for heating resistive element 133 which, in turn, heats edge 131a of substrate 131 to thermally cut tissue disposed between jaw members 110, 120, e.g., to cut the sealed tissue into first and second sealed tissue portions. Other configurations including multi-mode switches, other separate switches, etc. may alternatively be provided.

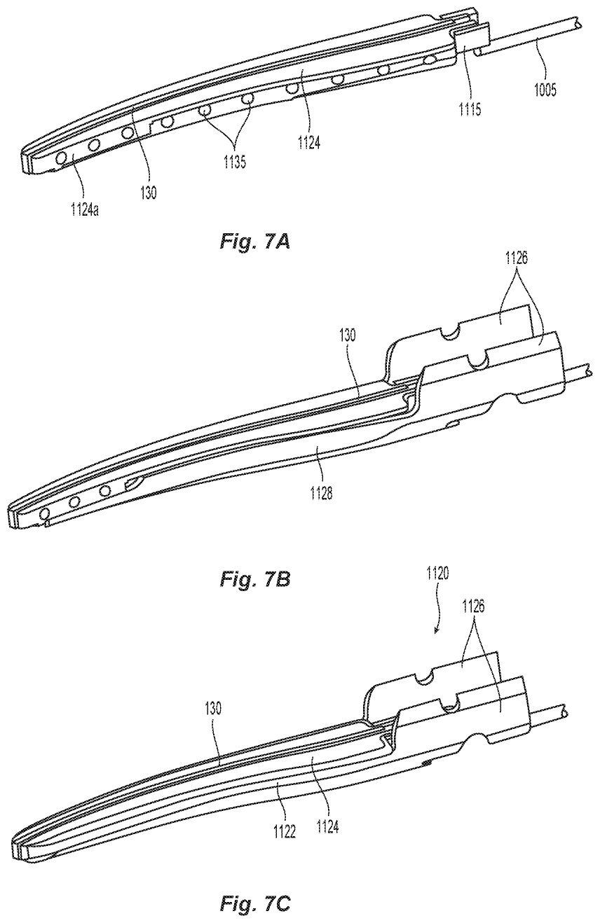

FIGS. 7A-7C show one embodiment of a jaw member 1120 for use with any of the aforementioned forceps 10, 210 described herein that utilizes a high temperature spacer 1124 to insulate the outer housing overmold 1122 from the high temperatures associated with thermal cutting. High temperature spacer 1124 may be manufactured from a material having a high temperature resistance and low thermal conductivity such as polybenzimidazole (PBI), e.g., a polybenzimidazole sold under the name Celazole® or other ceramic materials. More particularly, high temperature spacer 1124 of jaw member 1120 includes a generally flat upper surface configured to receive a conductive sealing surface (not shown) thereon or deposited thereon via one or more deposition processes, e.g., sputtering, which cooperates with an opposing surface (not shown) to seal tissue disposed therebetween when energized. During the deposition process, e.g., in particular, sputtering, the thermally and electrically conductive material(s) of the resistive element 133 is/are deposited on the substrate 131 and insulator 132 along a length thereof as mentioned above. The resulting thickness of the layer of resistive material (not shown) may be finely controlled with various deposition techniques onto flat surfaces, and, as a result, the temperature of the thermal cutter assembly 130 may be well-controlled.

A wire clip 1115 is configured to operably engage the spacer 1124 for ultimate connection to the conductive sealing surface (not shown). When a conductive material is applied to, e.g., deposited atop, the insulative spacer 1124 to form a tissue engaging, electrically conductive plate, e.g., 113, 123, the wire clip 1115 forms the electrical connection between the lead 1005 and the conductive material. Lead 1005 engages clip 1115 which, in turn, engages spacer 1124 on either side of a proximal end thereof (FIG. 7A) and ultimately electrically connects to the conductive material (as explained in detail above). During deposition, e.g., sputtering, onto the surface of the insulative spacer 1124 to form the electrically conductive, tissue engaging plate as discussed above, e.g., 113, 123, the conductive material may be deposited atop the wire clip 115 to form the electrical connection.

Spacer 1124 is configured to operably secure the thermal cutter assembly 130 prior to overmolding via a high temperature adhesive or other mechanical method of retaining the same. A plurality of spacers divots 1135 or other mechanical retention elements are disposed along one or more sides, e.g., side 1124a, of the spacer 1124 to operably receive and enhance the retention of the overmold 1122.

As shown in FIG. 7B, a jaw support 1128 operably engages the spacer 1124 on either side thereof to at least partially encapsulate the spacer 1124. The spacer divots 1135 may be configured to engage and enhance retention of the jaw support 1128 to the spacer 1124 or other mechanical retention features may be added to accomplish this purpose. Jaw support 1128 includes proximal flanges 1126 extending therefrom that are configured to engage the opposing jaw member, e.g., similar to jaw member 110, along with a pivot mechanism (not shown). Jaw support 1128 is also configured to encapsulate the wire clip 1115 (FIG. 7B). Once the jaw support 1128 is engaged with the spacer 1124, the jaw member 1120 is ready to accept the overmold 1122 via an overmolding process (See FIG. 7C). The high temperature spacer 1124 insulates the overmold 1122 from high temperatures that emanate from the thermal cutter assembly 130 when activated.

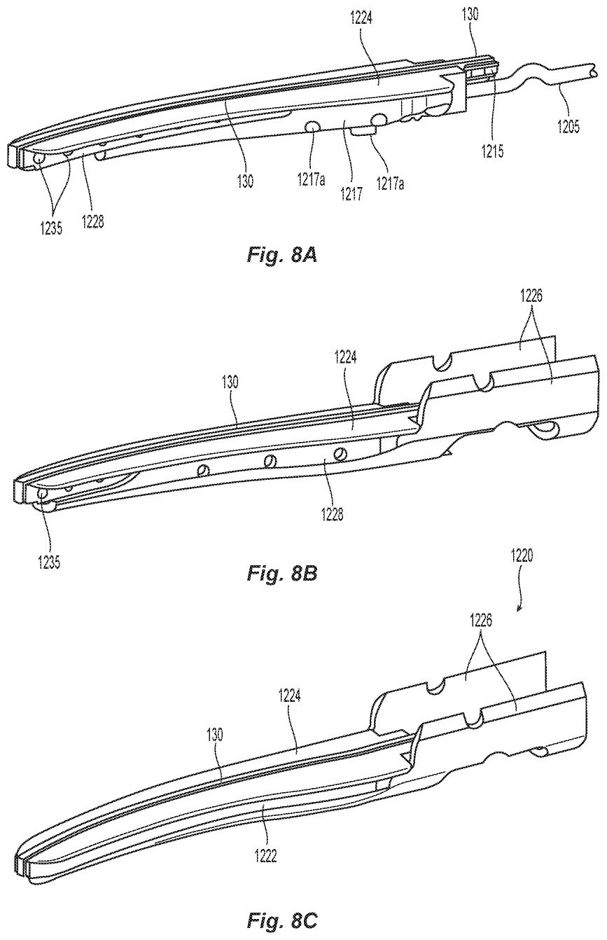

FIGS. 8A-8C show another embodiment of a jaw member 1220 for use with any of the aforementioned forceps 10, 210 described herein that utilize a high temperature spacer 1224 to insulate the outer housing overmold 1222 from the high temperatures associated with thermal cutting. In this embodiment, the spacer 1224 is held to the jaw member 1220 via a two-shot molding process.

High temperature spacer 1224 may be manufactured from a polybenzimidazole (PBI), e.g., a polybenzimidazole sold under the name Celazole® or other ceramic material. More particularly, high temperature spacer 1224 of jaw member 1220 includes a generally flat upper surface configured to receive a conductive material (not shown) thereon or deposited thereon via one or more deposition processes, e.g., sputtering, to form an electrically conductive, tissue engaging plate, e.g., plate 123, which cooperates with an opposing plate, e.g., plate 113 to seal tissue disposed therebetween when energized. A wire clip 1215 is configured to operably engage the spacer 1224 for ultimate connection to the conductive sealing plates 113, 123 (not shown).

Similar to FIGS. 7A-7C, spacer 1224 of FIGS. 8A-8C is configured to operably secure the thermal cutter assembly 130 prior to overmolding via a high temperature adhesive or other mechanical method of retaining the same. Lead 1205 engages clip 1215 which, in turn, engages spacer 1224 on either side of a proximal end thereof (FIG. 8A). A plurality of spacers divots 1235 or other mechanical retention elements are disposed along one or more sides, e.g., side 1224a, of the spacer 1224 to operably receive and enhance retention of the overmold 1222.

As shown in FIG. 8B, a first overmold 1217 partially engages the spacer 1224 near a proximal end thereof and includes mechanical features 1217a configured to operably engage the jaw support 1228. The jaw support 1228, in turn and when assembled, encapsulates the first overmold 1217 and a portion of the spacer 1224 therein. The spacer divots 1135 may be configured to engage and secure the jaw support 1228 to the spacer 1224 as well (or other mechanical retention features may be added to accomplish this purpose). Similar to the embodiment shown in FIGS. 7A-7C, jaw support 1228 includes proximal flanges 1226 extending therefrom that are configured to engage the opposing jaw member, e.g., similar to jaw member 110, along with a pivot mechanism (not shown). Jaw support 1228 is also configured to encapsulate the wire clip 1215 (FIG. 8B). Once the jaw support 1228 is engaged with the spacer 1224 and the first overmold 1217, the jaw member 1120 is ready to accept the second overmold 1222 via an overmolding process (See FIG. 8C). The high temperature spacer 1224 insulates the first and second overmolds 1217, 1222 from high temperatures that emanate from the thermal cutter assembly 130 when activated.

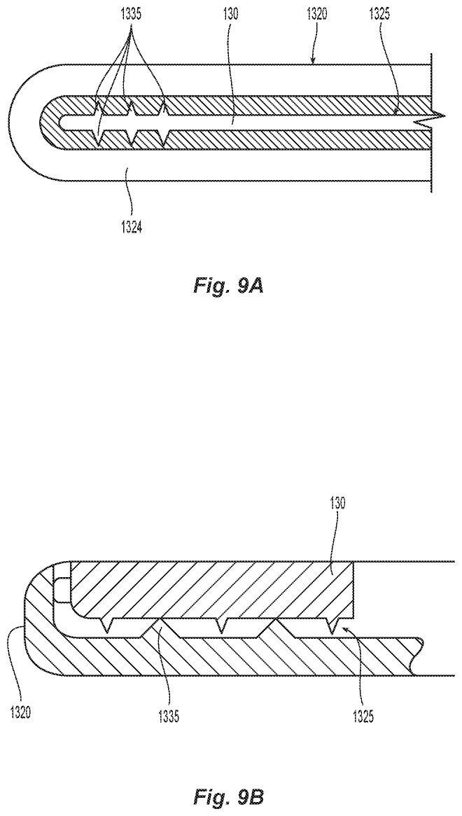

Turning now to FIGS. 9A and 9B, other embodiments to prevent unwanted thermal heat transfer to the outer overmolding include providing a series of standoffs 1335 along the inner peripheral a surface of a slot 1325 defined in the jaw member 1320. More particularly, jaw member 1320 includes a slot 1325 defined therein configured to operably receive thermal cutter assembly 130. The series of standoffs 1335 are disposed on the inner peripheral surface of the slot 1325 that are configured to extend therein to support and retain the thermal cutter assembly 130 within the slot 1325. The standoffs 1335 may be disposed in an alternating fashion along the slot 1325 (FIG. 9B) or may be disposed in registration with one another along the same (FIG. 9A) depending on a particular purpose. One or more standoffs 1335 may be disposed at a distal end of the slot 1325.

Standoffs 1335 are configured and dimensioned to minimize the direct contact between the thermal cutter assembly 130 and the spacer 1324 thereby reducing heat transfer to the other jaw components, e.g., jaw spacer 1324, jaw structure and/or the overmold (as described above). The standoffs 1335 may be made from any type of high temperature material known to reduce heat conduction, e.g., ceramic.

In other embodiments, the thermal cutter assembly 130 may be sprayed as a whole or along parts thereof with a material to create a thermal barrier between the thermal cutter assembly 130 and the surrounding jaw components, e.g., jaw spacer 1324, jaw structure and/or the overmold (as described above). Thermal materials such as Yttria-stabilized zirconia (YSZ) or other materials that have a low-thermal conductivity, and relatively high coefficient of thermal expansion may be utilized for this purpose.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A jaw member for a surgical instrument, comprising:
an insulative spacer configured to retain a thermal cutter assembly therein;
a jaw support configured to at least partially encapsulate the insulative spacer and securely engage the insulative spacer therein; and
an overmold configured to encapsulate and secure the insulative spacer and jaw support, wherein the insulative spacer is made from a material having a high temperature resistance and low thermal conductivity to reduce heat transfer to the overmold during activation of the thermal cutter assembly.

2. The jaw member according to claim 1, wherein the insulative spacer is made from a machinable ceramic material.

3. The jaw member according to claim 1, wherein the insulative spacer is made from polybenzimidazole (PBI).

4. The jaw member according to claim 1, wherein the thermal cutter assembly is retained within the insulative spacer by a high temperature adhesive.

5. The jaw member according to claim 1, further comprising a second overmold disposed between the jaw support and the insulative spacer.

6. The jaw member according to claim 1, wherein the insulative spacer includes a plurality of spacer divots configured to enhance operative engagement with the overmold.

7. A method of assembling a jaw member, comprising:
securing a thermal cutter assembly within a high temperature insulative spacer having a low conductivity;
encapsulating the insulative spacer with a jaw support, the jaw support including at least one proximal flange for operably engaging an opposing jaw member; and
overmolding the jaw support and insulative spacer to encapsulate and secure both the jaw support and the insulative spacer.

8. The method of assembling a jaw member according to claim 7, wherein the insulative spacer includes a plurality of spacer divots configured to enhance operative engagement with the overmold.

9. The method of assembling a jaw member according to claim 7, wherein prior to encapsulating the insulative spacer with the jaw support the method includes overmolding the insulative spacer to encapsulate and secure at least a portion of the insulative spacer.

10. The method of assembling a jaw member according to claim 7, wherein prior to encapsulating the insulative spacer with the jaw support the method includes engaging a wire to the insulative spacer.

11. The method of assembling a jaw member according to claim 10, wherein engaging the wire to the insulative spacer includes a wire clip.

12. The method of assembling a jaw member according to claim 7, wherein the method further includes depositing a conductive material atop a tissue engaging surface of the insulative spacer.

13. The method of assembling a jaw member according to claim 11, wherein the method further includes depositing a conductive material atop a tissue engaging surface of the insulative spacer and the wire clip to form an electrical connection between the conductive material and the wire.

14. The method of assembling a jaw member according to claim 7, wherein prior to encapsulating the insulative spacer with the jaw support the method includes overmolding the insulative spacer to encapsulate and secure at least a portion of the insulative spacer and engaging a wire to the insulative spacer.

15. The method of assembling a jaw member according to claim 14, wherein engaging the wire to the insulative spacer includes a wire clip.

16. The method of assembling a jaw member according to claim 15, wherein the method further includes depositing a conductive material atop a tissue engaging surface of the insulative spacer.

17. The method of assembling a jaw member according to claim 15, wherein the method further includes depositing a conductive material atop a tissue engaging surface of the insulative spacer and the wire clip to form an electrical connection between the conductive material and the wire.

18. The method of assembling a jaw member according to claim 17, wherein the conductive material is selected from the group consisting of aluminum, copper, chromium, titanium, stainless steel, nickel, chrome, tin, platinum, zinc, palladium, gold, nichrome, and ferritic iron-chromium-aluminum alloys.

19. The method of assembling a jaw member according to claim 7, wherein the insulative spacer is made from a machinable ceramic material.

20. The method of assembling a jaw member according to claim 7, wherein the insulative spacer is made from polybenzimidazole (PBI).
